(19) United States
(12) Patent Application Publication
(10) Pub. No.: US 2024/0425822 A1
(43) Pub. Date: Dec. 26, 2024

(54) LOCATION-MATCHED GROWTH MEDIA FORMULATIONS FOR THE DEVELOPMENT OF BRAIN TUMOR ORGANOIDS

(71) Applicant: Ann and Robert H. Lurie Children's Hospital of Chicago, Chicago, IL (US)

(72) Inventors: Milagros Melanie Suarez Palacios, Chicago, IL (US); Yuchen Du, Chicago, IL (US); Xiaonan Li, Chicago, IL (US)

(21) Appl. No.: 18/752,304

(22) Filed: Jun. 24, 2024

Related U.S. Application Data
(60) Provisional application No. 63/509,916, filed on Jun. 23, 2023.

(51) Int. Cl. C12N 5/09

(52) U.S. Cl. CPC C12N 5/0693; C12N 2500/32; C12N 2500/44; C12N 2500/90; C12N 2501/11; C12N 2501/115; C12N 2501/119; C12N 2501/13; C12N 2501/33; C12N 2501/41; C12N 2501/415; C12N 2501/59; C12N 2503/00

(57) ABSTRACT

Disclosed are formulations and methods for developing patient-derived brain cancer organoids, including site-specific patient-derived orthotopic xenograft (PDOX) organoids.

### CROSS-REFERENCE TO RELATED APPLICATIONS

This application claims priority to U.S. Provisional Application No. 63/509,916 filed on Jun. 23, 2023. The contents of which are incorporated by reference in their entireties.

### STATEMENT REGARDING FEDERALLY SPONSORED RESEARCH OR DEVELOPMENT

This invention was made with government support under grant number CA217613 awarded by the National Institutes of Health. The government has certain rights in the invention.

### FIELD OF INVENTION

The field of the invention relates compositions and methods for brain cancer research, including location-specific cancer cell culture media and organoids for use in brain cancer research.

### BACKGROUND

Brain cancer constitutes the most common type of childhood malignancies and the leading cause of death in children. Despite recent advances in diagnostic methods, surgical techniques as well as chemo- and radio-therapeutic strategies, the survival rates in many types of malignant pediatric brain tumors remain dismal, with <25% of children with high grade glioma surviving five years after initial diagnosis and nearly all patients with diffuse intrinsic pontine glioma succumbing to the disease within 9-12 months. Moreover, even among brain tumor survivors, many patients are left with long-term cognitive and/or neuroendocrine sequalae. Therefore, more efficacious therapeutic modalities that can improve the patient's quality of life and increase their survival are urgently needed.

Understanding tumor biology and developing new therapeutic drugs of brain cancers all need biologically accurate model systems. Organoids, which are composed of multiple types of tumor cells, normal brain cells and immune cells, represent the best in vitro model system. They replicate the three-dimensional structure of human tumors, can be rapidly produced (as compared with animal models), easily manipulated and mass produced for large scale drug testing. One unmet need of organoid development is that many patient tumors cannot form organoids. One of the major reasons is that the growth needs of tumors are not met, simply because different types of human cancers depend on distinct growth stimuli (i.e., growth factors) to proliferate. There is a need for improved in vitro systems for targeted brain tumor growth and replication of tumor biology.

### SUMMARY

An aspect of the disclosure is location-matched brain tumor culture formulations for producing location-derived organoids in specific locations including forebrain, midbrain, and hindbrain. The culture formulations contain growth factors required for culture of site-specific tissue or cells. One embodiment is a forebrain region tumor culture formulation that includes a basal medium supplemented with recombinant human basic fibroblast growth factor (FGF), recombinant human epidermal growth factor (EGF), brain-derived neurotrophic factor (BDNF), glial cell-derived neurotrophic factor (GDNF), neurotrophin 3 (NT-3), ciliary neurotrophic factor (CNTF), and wingless-relate integration site 3A (WNT-3A). Another embodiment is a midbrain region tumor culture formulation that includes a basal medium supplemented with recombinant FGF-8 and recombinant sonic hedgehog (SHH). Another embodiment is a hindbrain region (cerebellum, Pons) cancer cell culture formulation comprising a basal medium supplemented with recombinant human basic FGF, recombinant FGF-19, and recombinant stromal cell-derived factor 1 alpha (SDF-1-α). A further embodiment is a hindbrain region (brainstem) cancer cell culture formulation comprising a basal medium supplemented with recombinant human basic FGF, recombinant human EGF, BDNF, GDNF, and NT-3.

Another aspect of the disclosure is forming brain region tumor site-specific organoids. Disclosed are methods of forming brain region tumor organoids including producing a patient-derived orthotopic xenograft (PDOX) with forebrain region tumor, midbrain region tumor, or hindbrain region tumor, and culturing the PDOX tumor in the disclosed location-matched formulations to form specific brain region patient-derived xenograph organoids (PDXOs). Further methods include producing a patient-derived orthotopic xenograft (PDOX) with specific brain region tumor and culturing the PDOX tumor in the matching location-specific culture formulation to generate patient-derived xenograft organoids (PDXO), which are location- or site-specific.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

It should be understood that the drawings described below are for illustration purposes only and are not intended to limit the scope of the present teachings in any way.

FIG. 1 illustrates matching panels of animal and organoid model systems for pediatric brain tumors.

FIGS. 2A-2B illustrate orthotopic implantation of patient brain tumor cells into matched locations in mouse brains for the development of patient-derived orthotopic xenograft (PDOX) models. For supratentorial tumors such as GBM, a burr hole is made on the right parietal bone 1 mm to the midline and 2 mm anterior to the bregma occipital line for intra-cerebral (IC) implantation. For infra-tentorial tumors such as medulloblastoma, the burr hole is made on the right inter-parietal bone 1 mm to the midline and 1 mm posterior to the bregma occipital line for intra-cerebellar (ICb). For brain stem tumors, the burr hole is made at the right corner of the midline and bregma occipital line for intra-brain stem (IBs) injection. Tumor cells are injected at a depth of 3 mm below the outer surface of the skull for IC (near the right caudate nucleus) and ICh (in the middle of the right hemisphere of the cerebellum) and 5.2 mm for IBs (in the middle of pons) implantation (FIG. 2A). Brain images show xenograph development (FIG. 2B).

FIGS. 3A-3F show generation of organoids from patient samples and patient-derived orthotopic xenograft (PDOX) models. Exemplary steps for patient tumor or patient-derived orthotopic xenograft tumor collection in DMEM/F12 (FIG. 3A), include tissue transfer to a petri dish before cutting (FIG. 3B), mechanical mincing with sterile scissors (FIG. 3C), dissociation (FIG. 3D), cell suspension (FIG. 3E), and transfer of cell suspension to 24-well culture plates coated with agar (FIG. 3F).

FIGS. 4A-4C illustrate organoid formation from patient samples facilitated by site-related media. Cerebral tumor resulted in 85.7% organoid formation (6 out of 7) (FIG. 4A), cerebellar tumor resulted in 100% (4 out of 4) (FIG. 4B), and brain stem tumor from biopsy, resulted in 100% (2 out of 2) (FIG. 4C). Total organoid formation for all models was 90.9% (20 out of 22). Patient-derived organoids from cerebral tumor are given identifiers in Table 8.

FIGS. 5A-5C illustrate organoid formation from cerebral PDOX tumor samples facilitated by site-related media. Cerebral PDOX tumors resulted in 87.5% organoid formation (21 out of 24) (FIGS. 5A-5C). Patient-derived cerebral PDOX organoids (PDXOs) are given identifiers in Table 9.

FIGS. 6A-6C illustrate organoid formation from cerebellar PDOX tumor samples facilitated by site-related media. Cerebellar PDOX tumors resulted in 75% organoid formation (12 out of 16) (FIGS. 6A-6C). Patient-derived cerebellar PDXOs are given identifiers in Table 10.

FIGS. 7A-7B illustrate organoid formation from brainstem PDOX tumor samples facilitated by site-related media. Brainstem PDOX tumors resulted in 77.7% organoid formation (7 out of 9) (FIGS. 7A-7B). Brainstem PDXOs are given identifiers in Table 11.

FIGS. 8A-8R illustrate cellular characterization studies of PDOX-derived organoids that reveal replication of a heterogenous population. Hematoxylin and Eosin (H&E) staining for brainstem region models IBs-9119DIPG (FIG. 8A) and IBs-A0317PNET (FIG. 8E). Hematoxylin and Eosin (H&E) staining for cerebral models IC-G0606GBM (FIG. 8I) and IC-2305GBM (FIG. 8L). Immunohistochemical staining for brainstem region models IBs-9119DIPG of Ki67 (FIG. 8B), Caspase-3 (FIG. 8C), and H3K27me3 (FIG. 8D), and IBs-A0317PNET of Ki67 (FIG. 8F), Caspase-3 (FIG. 8G), and H3K27me3 (FIG. 8H). Immunohistochemical staining for cerebral samples IC-G0606GBM of Ki67 (FIG. 8J) and Caspase-3 (FIG. 8K), and IC-2305GBM of Ki67 (FIG. 8M), and Caspase-3 (FIG. 8N). Immunofluorescence staining for cerebral sample (FIGS. 8O-8P) and cerebellar sample (FIG. 8Q-8R) of mitochondria (green), cytoplasm (red), lysosome (red) and nuclei (blue).

FIGS. 9A-9C illustrate single-cell RNA sequencing data of parental tumors and PDOX-derived organoids that demonstrate maintenance of cellular heterogeneity and molecular signatures of parental tumors. UMAP dimensionality reduction plot shows the cluster distribution of cells obtained from parental tumors (2691-PDOX, 2691-Xeno, 3752-Xeno, 9119-PDOX) and corresponding PDOX-derived organoid samples (2691-GBM, 3752-GBM, 9119-DIPG) (FIG. 9A). UMAP plot shows the different independent clusters obtained by integrating the malignant cells from parental tumors and corresponding PDOX-derived organoids (FIG. 9B). Heatmap of gene expression of clusters identified in parental tumors and corresponding PDOX-derived organoid samples with hierarchical clustering by Euclidian distance (FIG. 9C).

### DETAILED DESCRIPTION

The descriptive embodiments in this disclosure are not limited to particular methods, reagents, compounds, compositions or biological systems, which can, of course, vary. It is also to be understood that the terminology used herein is for the purpose of describing particular embodiments only, and is not intended to be limiting.

The terms “a,” “an,” “the” and similar references in this disclosure, including in the context of the claims, include both the singular and the plural, unless indicated otherwise or the context clearly indicates otherwise.

Further, ordinal indicators—such as “first,” “second,” “third,” etc.—for identified elements are used to distinguish between the elements, and do not indicate or imply a required or limited number of such elements, and do not indicate a particular position.

All methods described herein can be performed in any suitable order unless otherwise indicated herein or otherwise clearly contradicted by context.

The use of any and all examples, or exemplary language (e.g., “such as”) provided herein is intended merely to illuminate a description or embodiment and does not pose a limitation on the scope of the invention otherwise claimed. No language in the present specification should be construed as indicating any non-claimed element essential to the practice of the invention.

Also as used herein, “and/or” refers to and encompasses any and all possible combinations of one or more of the associated listed items, as well as the lack of combinations when interpreted in the alternative (“or”).

Unless stated otherwise, the term “about” refers to an approximation of a stated value within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “patient” or “subject” or “individual” may be used interchangeably, and refers to all animals, including mammals, e.g., a human or a non-human mammal, who are prone to or suffering from the indicated disease or disorder, or who are treated with [the pharmaceutical compositions or in accordance with the methods described herein].

A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

The term “diagnosis” refers to a relative probability a subject has a given disorder. Similarly, the term “prognosis” refers to a relative probability that a certain future outcome may occur in the subject. For example, in the context of the present disclosure, prognosis can refer to the likelihood that an individual will develop a brain tumor. Prognosis can also refer to the likely severity of the disease (e.g., severity of symptoms, rate of functional decline, etc.). The terms are not intended to be absolute, as will be appreciated by any one of skill in the field of medical diagnostics.

As used here, “cancer” refers to diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that begins in blood-forming tissue, such as the bone marrow, and causes too many abnormal blood cells to be made. Lymphoma and multiple myeloma are cancers that begin in the cells of the immune system. Central nervous system cancers are cancers that begin in the tissues of the brain and spinal cord. Also called malignancy.

In some embodiments, the subject may be diagnosed with, or suspected of having a brain cancer or a brain tumor. The brain cancer or tumor may be of any type or origin, and may be present in pediatric or adult subjects. In some embodiments the brain tumor may be an embryonal tumor, a neuroblastoma, a glioma, an ependymoma, an optic nerve glioma, a craniopharyngioma, glioblastoma, meningioma, pituitary tumor, vestibular schwannoma, medulloblastoma, choroid plexus tumor, pineal tumor, chordoma, chondrosarcoma, olfactory neuroblastoma, astrocytoma, ependymal tumor, hemangiopericytoma a germ cell tumor, oligodendroglioma, brain metastases or a ganglioglioma. In the Examples, the inventors developed models of pediatric and adult brain cancers. The inventors tested diffuse intrinsic pontine glioma (DIPG), low-grade glioma (LGG), medulloblastoma (MB), high-grade glioma (HGG), atypical teratoid rhabdoid tumor (ATRT), astrocytomas, primitive neuro-ectodermal tumors (PNETs) and glioblastoma (GBM).

Pediatric brain tumor biology is known to be determined/related to tumor location. Similarly, the growth of brain tumoral organoids is expected to be dependent on tumor location-related niche factors. Previous studies on brain organoid formation from pluripotent stem cells have suggested a strong dependence on growth factor combinations unique/selective to different brain regions. Therefore, a new set of formulations of growth media (with distinct growth factor composition) was developed for tumors originated from various regions of the brain, including the forebrain (cerebral cortex), the midbrain region (tectum and cerebral peduncle), hindbrain region (cerebellum, pons), and brainstem. These new formulations are used for developing patient-derived cancer organoids that can provide high fidelity to human cancers for research, and significantly increase the predictability of preclinical therapy options.

Disclosed are formulations and methods for developing patient-derived cancer organoids. The cancer organoids may be developed from a patient tumor sample from specific cancer site, for example brain cancer. Organoids may be generated from site-specific patient cancer samples, or from location-matched patient-derived orthotopic xenograft (PDOX). Orthotopic xenograft refers to a process of implanting human cancer cells in an animal, for example a mouse, into the same organ or tissue from which the cancer originated in the human donor.

### Formulations

Exemplary models for patient tumor or patient-derived orthotopic xenograft tumor organoid development include use of location-matched cell culture formulation for collection and culture of brain tumor tissue (patient tumor or patient-derived orthotopic xenograft). Location-specific or site-specific formulations include a basal medium supplemented with site-required growth factors. The growth factors are unique and selective to the various sites or regions of the brain for region development. The different regions of the brain include forebrain (cerebrum), midbrain (tectum and cerebral peduncle), hindbrain (cerebellum, pons), and hindbrain (brainstem).

To generate specific brain region tumor organoids, patient tumor samples or patient-derived orthotopic xenograft tumor from specific brain regions are collected in basal medium supplemented with site-required growth factors. An exemplary base medium is composed of serum-free DMEM/F12 Media, with additional components including B27, N2, MEM-NEEAs, Penicillin-Streptomycin, GlutaMAX, 2-mercaptocthanol, and human insulin. Different growth factor combinations/cocktails are added to this base media, according to the region in which the tumor developed. Brain region location-matched culture media are supplemented for growth of tumor cells originated from various regions of the brain, including the forebrain (cerebral cortex), the midbrain region (tectum and cerebral peduncle), hindbrain region (cerebellum, pons), and brainstem, which require distinctly different factors for growth. One of skill in the art can recognize alternatives to some elements of the base media may be used. For example, antibiotics used may include penicillin-streptomycin and/or gentamicin, and amino acids used may include L-glutamine and/or GlutaMAX.

In embodiments, a formulation for a forebrain region tumor or cancer cell culture may include the base medium supplemented with fibroblast growth factor (FGF), epidermal growth factor (EGF), brain-derived neurotrophic factor (BDNF), glial cell-derived neurotrophic factor (GDNF), neurotrophin 3 (NT-3), ciliary neurotrophic factor (CNTF), laminin, and wingless-relate integration site 3A (WNT-3A. In some embodiments the FGF is recombinant FGF, such as recombinant human FGF-basic. In some embodiments, the EGF is recombinant EGF, such as recombinant human EGF protein. In some embodiments, laminin is included. In the embodiments, the forebrain region tumor or cancer cell is cerebral cortex tumor or cancer cell. Recombinant FGF, recombinant FGF, recombinant EGF, BDNF, GDNF, NT-3 may be used in a range of about 2 ng/ml to about 200 ng/ml, preferably around 20 ng/ml. CNTF and WNT-3A may be used in a range of about 1 ng/ml to about 100 ng/ml, preferably about 10 ng/ml.

In embodiments, a formulation for a midbrain region tumor or cell culture medium may include the base medium supplemented with FGF-8 and sonic hedgehog (SHH) protein. In some embodiments, the FGF-8 is recombinant FGF-8, for example recombinant human FGF-8. In some embodiments, the SHH is recombinant SHH, such as recombinant human SHH. Recmonbinant FGF-8 and recombinant SHH may be used in a range of about 10 ng/ml to about 1000 ng/ml, preferably about 100 ng/ml. In the embodiments, the midbrain region tumor or cancer cell is tectum tumor or cerebral peduncle tumor.

In embodiments, a formulation for a hindbrain region tumor or cell culture medium may include the basal medium supplemented with FGF, FGF-19, and stromal cell-derived factor 1 alpha (SDF-1-α). In some embodiments the FGF is recombinant basic FGF, for example recombinant human FGF-basic. In some embodiments, the FGF-19 is recombinant FGF-19, for example recombinant human FGF-19. In some embodiments, the SDF-1-α is recombinant SDF-la, such as recombinant human SDF-1-α. In the embodiments, the hindbrain region tumor or cancer cell is cerebellum tumor or pons tumor. Recombinant FGF-basic may be used in a range of about 5 ng/ml to about to about 500 ng/ml, preferably about 50 ng/ml. Recombinant FGF-19 may be used in a range of about 10 ng/ml to about 1000 ng/ml, preferably about 100 ng/ml. Recombinant SDF-la may be used in a range of about 30 ng/ml to about 3000 ng/ml, preferable about 300 ng/ml.

In embodiments, a formulation for a hindbrain region culture medium may include the basal medium supplemented with FGF, EGF, BDNF, GDNF, NT-3. In some embodiments the FGF is recombinant basic FGF, including recombinant human FGF-basic. In some embodiments, the EGF is recombinant EGF, including recombinant human EGF protein. Recombinant FGF-basic, recombinant EGF protein, BDNF<GDNF and NT-3 may be used in a range of about 2 ng/ml to about 200 ng/ml, preferably 20 ng/ml. In the embodiments, the hindbrain region tumor or cancer cell is brainstem tumor.

### Methods of Use

The region-specific formulations allow production of location-matched tumor organoids. Disclosed are methods of organoid formation from patient-derived forebrain (cerebrum), midbrain (tectum and cerebral peduncle), hindbrain (cerebellum, pons), or hindbrain (brainstem) tumor samples facilitated by site-related media formulations.

In some embodiments, the method comprises using primary patient tumor cells. The patient tumor cells can be collected by any means known in the art, including biopsy, surgery, or from biological fluids or sample such as serum, blood, urine, amniotic fluid, plasma, saliva, cerebrospinal fluid, bone marrow or cells. Primary patient tumor cells may be cultured with the appropriate media described herein to generate an organoid, or patient derived organoid. For example, tumor cells collected from a cerebellar tumor can be cultured in the disclosed cerebellar media to generate a patient derived organoid. As described herein, primary patient tumor cells are dissociated into single cells which are transferred to culture vessel, such as an agar-coated plate, with a serum-free base media and location-matched growth factors or location matched media to generate patient derived organoids. Organoids are maintained in sterile culture at 37° C. 5% CO₂ for one week, 2 weeks, 3 weeks, 4 weeks, 1 month, 2 months, 3 months, 4 months, 5 months or 6 months. Preferably organoids are cultured for 4 months or less. In some embodiments 10,000 to 500,000 tumor cells are transferred into the culture plate. In preferred embodiments, 50,000 to 100,000 tumor cells are transferred.

To generate patient-derived xenograft organoids (PDXO), patient tumor cells are collected and injected into an animal model that allows the injected tumor cells to grow. For example, the patient tumor cells may be placed into an immunocompromised an animal, for example a mouse, such as a severe combined immunodeficiency disease (SCID) mouse, which will allow the patient tumor to grow and create a patient-derived orthotropic xenograft (PDOX). Tumor cells from the PDOX can then be collected and dissociated into single cells, transferred to culture vessel or plate, such as an agar-coated plate, and grown in serum-free base media and location-matched growth factors or location matched media to generate patient-derived xenograft organoids (PDXO). Organoids are maintained in sterile culture at 37° C. 5% CO₂ for one week, 2 weeks, 3 weeks, 4 weeks, 1 month, 2 months, 3 months, 4 months, 5 months or 6 months. Preferably organoids are cultured for 4 months or less. In some embodiments 10,000 to 500,000 tumor cells are transferred into the agar-coated plate. In preferred embodiments, 50,000 to 100,000 tumor cells are transferred.

Further methods include cellular characterization of the organoids to identify the cell population produced. Cellular characterization using histopathology identify specific PDOX-derived organoids. In embodiments, the PDOX-derived organoids reveal replication of a heterogenous population in the organoids.

The in vitro tumor organoid models developed herein can replicate the in vivo biology of patient tumors and allow for direct transition to in vivo clinical study.

The invention can be further understood in view of the following non-limiting examples.

### EXAMPLES

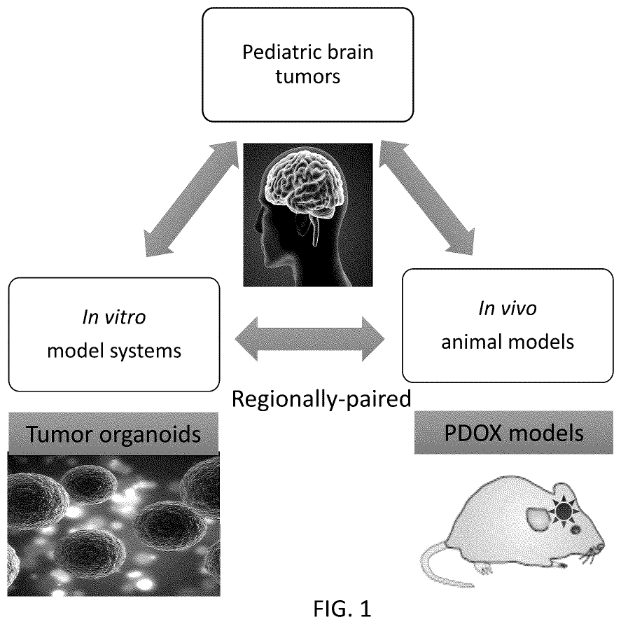

The majority of biological and pre-clinical studies on pediatric brain tumors are initiated in in vitro model systems before being validated in vivo in animal models. Therefore, the use of regionally-paired models derived from a common patient would greatly facilitate direct transition of in vitro findings to in vivo discovery and maximize the chances of future clinical success. Organoids are one of the best in vitro model systems as they preserve a tumor's cancer stem cell (CSC) pools and maintain a 3D structure with multiple cellular components similar to those found in the original patient tumors. (FIG. 1)

For pediatric brain tumors, however, few organoid models have been developed primarily due to limited supply of patient tumor cells and a low success rate. Artificially printed (3D printing) organoids have not increased the success of producing tumor organoids, due to limited starting materials and tumor cells are not grown in a “natural” manner. Hence, there is poor replication of tumor biology. One of the major reasons why most patient tumors do not form organoids is that the growth needs of tumors are not met, simply because different types of human cancers depend on distinct growth stimuli (i.e., growth factors) to proliferate. Using one-size-fit-all growth medium can therefore have limited success rate (0%-50%).

An alternative approach is disclosed that establishes a xenograft model of patient tissue and uses this tissue to generate specific tumor organoids to replicate the in vivo biology of patient tumors. Recognizing the impact of the tumor microenvironment and the brain developmental stage on brain tumor growth, a novel protocol was developed to evaluate the impact of tumor location-related niche factors on the development of brain tumoral organoids. Utilizing different growth factor combinations that are unique and selective to different regions of brain development, PDOX-derived and patient-derived organoids were developed. Specifically, 40 PDOX-derived organoids from a total of 49 PDOX models tested (success rate of 81.6%) and 20 patient-derived organoids from a total of 22 patient samples tested (success rate of 90.9%) were successfully produced.

Moreover, cellular characterization studies revealed the replication of a heterogenous cell population. Altogether, differences on growth factor dependence for the formation of organoids from brain tumors of different locations are identified, and PDOX models are demonstrated to be valuable tools for the development and optimization of protocols for organoid model establishment.

### Example 1. PDOX-Derived Cancer Organoid Models

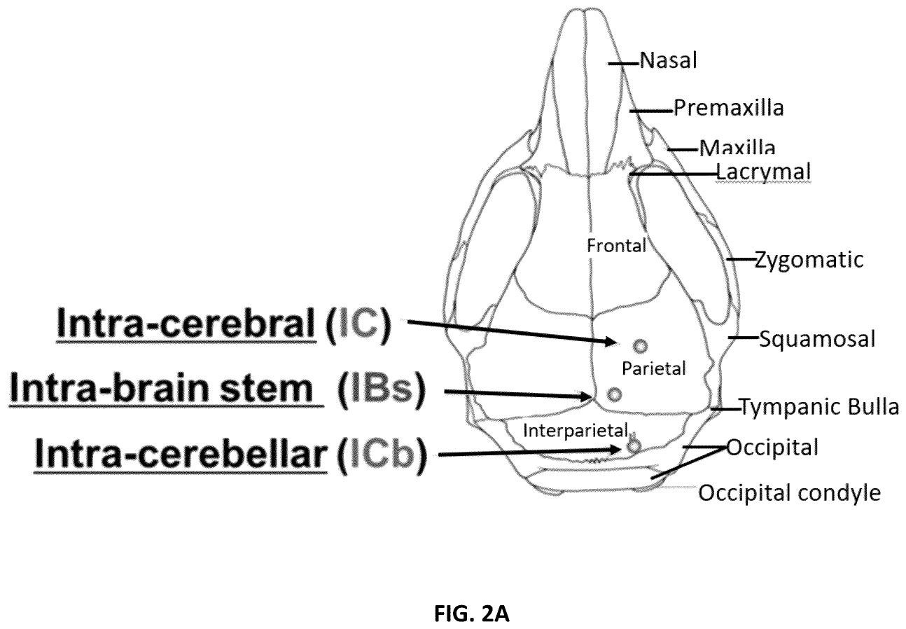

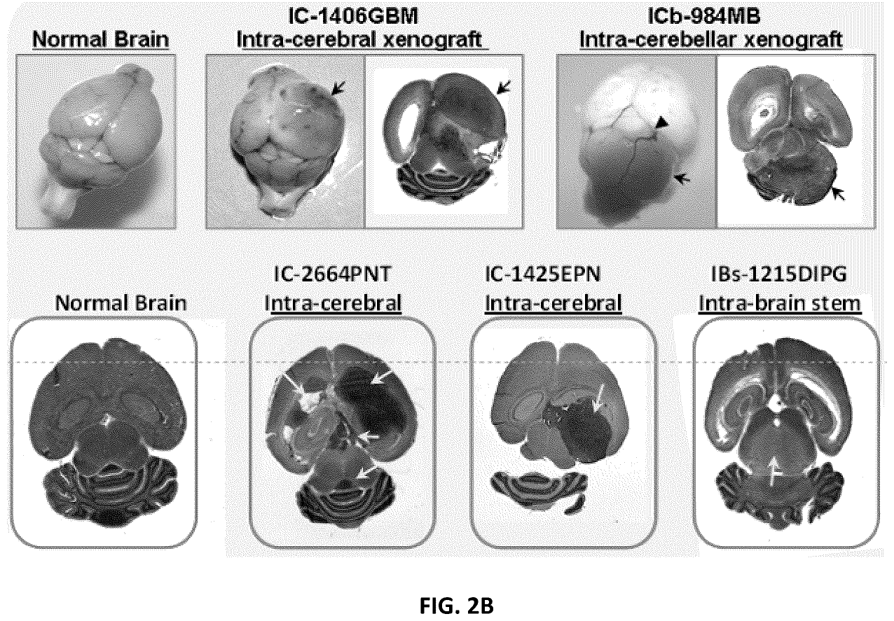

A new approach is developed to establish a xenograft model of patient tissue and use this tissue to generate organoids. (FIG. 2). These organoids receive the name of patient-derived xenograft organoids (PDXOs) and have been shown to replicate the in vivo biology of patient tumors. Compared with in vivo models, PDXOs are relatively easier and faster to establish. This means that PDXOs are better suited to large scale studies (testing multiple agents and combinations) than patient-derived xenografts (PDOXs). Moreover, by increasing the cohorts of tumor-bearing mice, the tumor cell supply is theoretically unlimited to meet the need of even the most comprehensive biological studies and high-throughput drug screenings.

For PDOX model development, patient pediatric brain tumor samples from specific brain sites (cerebrum, cerebellum, brain stem) were implanted into matching locations of severe combined immunodeficient (SCID) mice. A microsurgical drill is used to create a Burr hole (0.7 mm in diameter) in each specific site, with a depth of 3 mm for intra-cerebrum and intra-cerebellum, and 5.2 mm for intra-brain stem. A cell suspension of 2 μL is used for intra-cerebrum and intra-cerebellum, and 1 μL for intra-brain stem, using a 10 μl Hamilton syringe. (FIG. 2A). The same location in each brain had the same depth burr hole and had the same volume of cells implanted. 150 PDOX models were developed (FIG. 2B). Table 1 below lists the specific brain tumor-site samples used for PDOX models.

TABLE 1
PDOX models of pediatric brain tumor

| | Clinical Stage | | | |
|---|---|---|---|---|
| Tumor type | At diagnosis | Relapse | Terminal * | Subtotal |
| Medulloblastoma | 46 | 3 | 5 | 54 |
| Glioblastoma | 15 | 6 | 11 | 32 |
| Ependymoma | 13 | 10 | 1 | 24 |
| DIPG | | | 15 | 15 |
| ATRT | 7 | 1 | 2 | 10 |
| ETMR | 3 | | 1 | 4 |
| CNS-Germinoma | | 2 | | 2 |
| CNS-EFT-CIC | 1 | 2 | | 3 |
| PXA | 1 | | 1 | 2 |
| Others | 4 | | | 3 |
| Total | 90 | 24 | 36 | 150 |

* from autopsy; DIPG-diffuse intrinsic pontine glioma; ATRT = atypical teratoid/rhabdoid tumor; ETMR-embryonal tumor with multilayered rosettes; CNS EFT- CIC = ewing sarcoma family with CIC alteration; PXA = pleomorphic xanthosarcoma.

### Example 2. Customized Organoid Media for the Development of Organoids from Different Brain Locations

Recognizing the architecture and cellular complexity of the developing brain as well as the dependance of distinct human cancers on distinct growth stimuli to proliferate, a customized growth media was developed with specific growth factor formulations that have been previously shown to be involved in guiding neural progenitor fate specification during mammalian brain development in order to support the growth of organoids from tumors originated from different brain regions including the cerebrum, cerebellum, and brain stem. Table 2 presents the base medium formulation with the concentration for each component.

TABLE 2
Base Media Formulation

Serum-free media (DMEM/F12)
B-27 (1X)
N2 (1X)
NEEAs (1X)
Penicillin-Streptomycin (1X)
GlutaMAX (1X)
2-mercaptoethanol (1X)
Human insulin (2.5 mg/mL)

Table 3 illustrates region-relevant small-molecules and growth factors tested in brain organoid formation from pluripotent stem cells. Previous studies have identified distinct combinations of small-molecules and growth factors that are specific/selective to different brain locations during fetal brain development (Table 3), which has provided important start points to guide the selection of growth factor combinations for the in vitro analysis.

TABLE 3
Region-relevant small molecules and growth factors tested in brain organoid formation from pluripotent stem cells.

| Week 4 | Week 5 | | | |
|---|---|---|---|---|
| (pregnancy) | (pregnancy) | Adult | Growth factor combination ^{(reference)} | |
| Prosencephalon | Telencephalon | Rhinencephalon, Amygdala, Hippocampus, | 1) | FGF2¹⁻³ |
| (forebrain) | Diencephalon | Cerebrum (Cortex), Hypothalamus, Pituitary Basal | 1) | OTX2, FoxG, EMX1, Nkx2.1⁴ |
| Mesencephalon | Mesencephalon | Ganglia, lateral ventricles | 1) | BDNF, GDNF, NT-3, |
| (midbrain) | Metencephalon | Epithalamus, Thalamus, Subthalamus, Pineal, 3^{rd} | | Laminin, bFGF⁶ |
| Rhomb encephalon | Myelencephalon | ventricle | 1) | DKK1, BMPRla-FC⁶ |
| (hindbrain) | | Tectum, Cerebral peduncle, | 2) | FGF2, EGF, BNDF, NT3⁷ |
| | | Pretectum, cerebral aqueduct | 1) | FoxA2, Pax5⁴ |
| | | Pons, Cerebellum | 1) | HoxB2, HoxA4, HoxB4, |
| | | Medulla Oblongata | | HoxG6⁴ |
| | | | 2) | SHH (medulloblastoma) |
| | | | 3) | WNT (medulloblastoma) |

Site specific media developed for the forebrain region (cerebral cortex), midbrain region (tectum, cerebral peduncle), hindbrain region (cerebellum, pons), and brainstem are presented in Tables 4-7 below, with concentration used for each growth factor.

TABLE 4
Culture Formulation for Forebrain Region

| Forebrain Region (Cerebral Cortex) | ng/mL |
|---|---|
| Recombinant Human FGF-basic (R&D systems) | 20 |
| Recombinant Human EGF Protein (R&D systems) | 20 |
| BDNF (Peprotech) | 20 |
| GDNF (Peprotech) | 20 |
| NT-3 (Peprotech) | 20 |
| CNTF (Peprotech) | 10 |
| WNT-3A (R&D Systems) | 10 |

TABLE 5
Culture Formulation for Midbrain Region

| Midbrain Region (Tectum, Cerebral peduncle) | ng/mL |
|---|---|
| Recombinant Human FGF-8 (Peprotech) | 100 |
| Recombinant Human SHH (Peprotech) | 100 |

TABLE 6
Culture Formulation for Hindbrain Region (Cerebellum, Pons)

| Hindbrain (Cerebellum, Pons) | ng/mL |
|---|---|
| Recombinant Human FGF-basic (R&D systems) | 50 |
| Recombinant Human FGF-19 (Peprotech) | 100 |
| Recombinant Human SDF-1a (Peprotech) | 300 |

TABLE 7
Culture Formulation for Hindbrain Region (Brainstem)

| Hindbrain (Brainstem) | ng/ml |
|---|---|
| Recombinant Human FGF-basic (R&D systems) | 20 |
| Recombinant Human EGF Protein (R&D systems) | 20 |
| BDNF (Peprotech) | 20 |
| GDNF (Peprotech) | 20 |
| NT-3 (Peprotech) | 20 |

### Example 3 Generation of Organoids from Patient Samples and Patient-Derived Orthotopic Xenograft Models

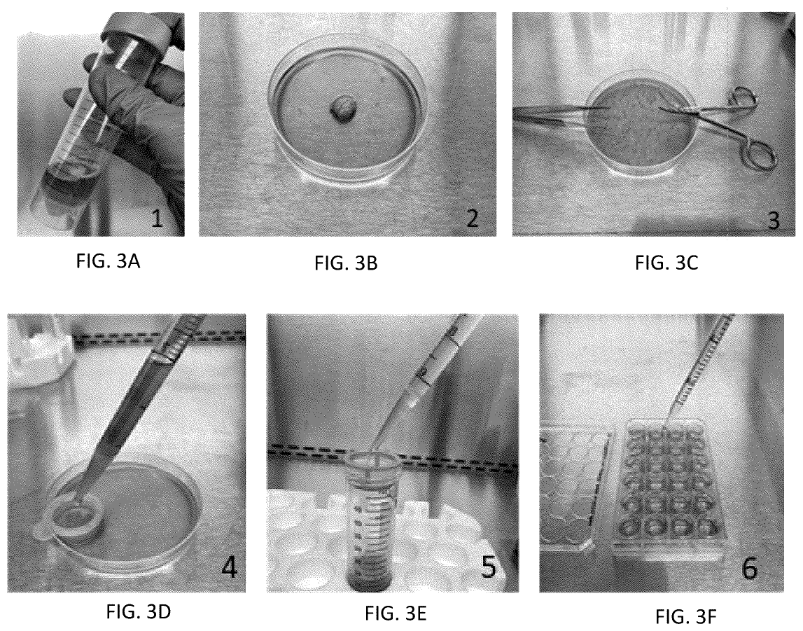

Three dimensional (3D) organoid cultures have arisen as clinically relevant and molecularly accurate in vitro brain tumor model systems for preclinical drug testing. These models have been demonstrated to recapitulate the genomic and phenotypic complexities of the original tumor and thereby, can reliably predict the patient response to treatment. Here, organoids are generated from patient samples and patient-derived orthotopic xenograft (PDOX) models. Xenograft tumors were collected in DMEM/F12 (FIG. 3A), and tissue transferred to a petri dish before cutting (FIG. 3B). Tissue was mechanically minced with sterile scissors (FIG. 3C), and dissociated (FIG. 3D). The resulting cells were suspended in media (FIG. 3E), and transferred to 24-well culture plates coated with agar (FIG. 3F). Organoid cells were then characterized by immunohistology and single-cell RNA sequencing.

### Example 4 Site-Related Media Facilitate Organoid Formation from Patient Samples

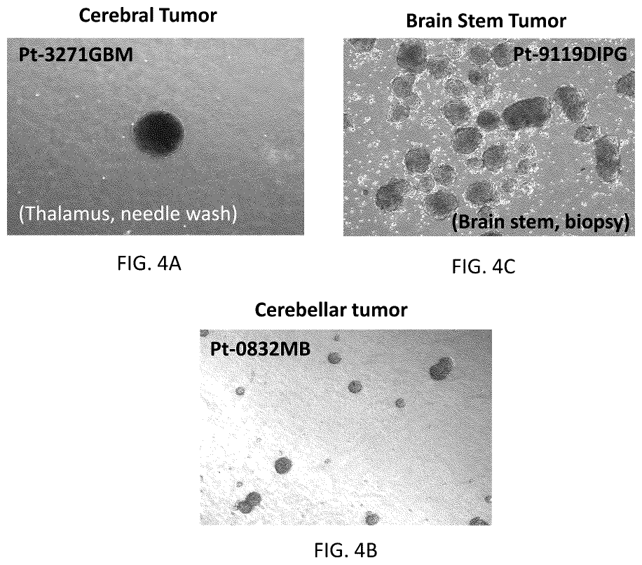

Using site-specific media in Example 2, cerebral cortex tumor (Pt-3271 GBM, thalamus sample from needle wash) resulted in 85.7% organoid formation (6 out of 7) (FIG. 4A). Cerebellar tumor (Pt-0832 MB) resulted in 100% (4 out of 4) organoid formation (FIG. 4B). Brainstem tumor from biopsy (Pt-9119DIPG) resulted in 100% (2 out of 2) formation. A total of 90.9% (20 out of 22 samples) resulted in organoid formation. Table 2 below lists the patient sample model identifier for FIG. 4A-4C.

TABLE 8
Model IDs and Tumor Sites for FIG. 4

| Model ID for FIG. 4 | Tumor Site |
|---|---|
| Pt-3759 (Posterior fossa) | Hindbrain (cerebellum, pons) |
| Pt-3271DMG | Midbrain (thalamus) |
| Pt-2959MB | Hindbrain (cerebellum, pons) |
| Pt-2493DIPG | Hindbrain (brainstem) |
| Pt-3068AST | Forebrain (cerebrum) |
| Pt-0900PA | Forebrain (cerebrum) |
| Pt-4276AST | Forebrain (cerebrum) |
| Pt-0700PA | Forebrain (cerebrum) |

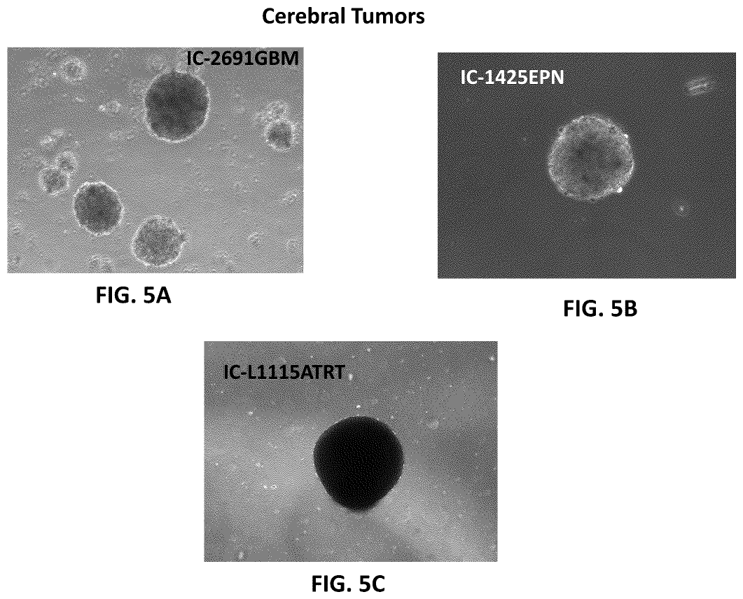

Brightfield images of organoids derived from single samples of cerebral PDOX tumors (FIGS. 5A-5C). Cerebral PDOX-derived organoids' identifiers are listed in Table 3. Images of individual organoids were taken every day using the Keyence microscope and Zen (Carl Zeiss) software.

TABLE 9
Cerebral Tumor Model IDs for FIG. 5

IC-A122GBM
IC-A46GBM
IC-1425EPN
IC-8100GBM
IC-2305GBM
IC-1406GBM
IC-NBRX121GBM
IC-9419PA
IC-L1115ATRT
IC-2691GBM
IC-3752GBM
IC-2644PNET
IC-G0606GBM
IC-K014/33/34/35/36/60/64GBM

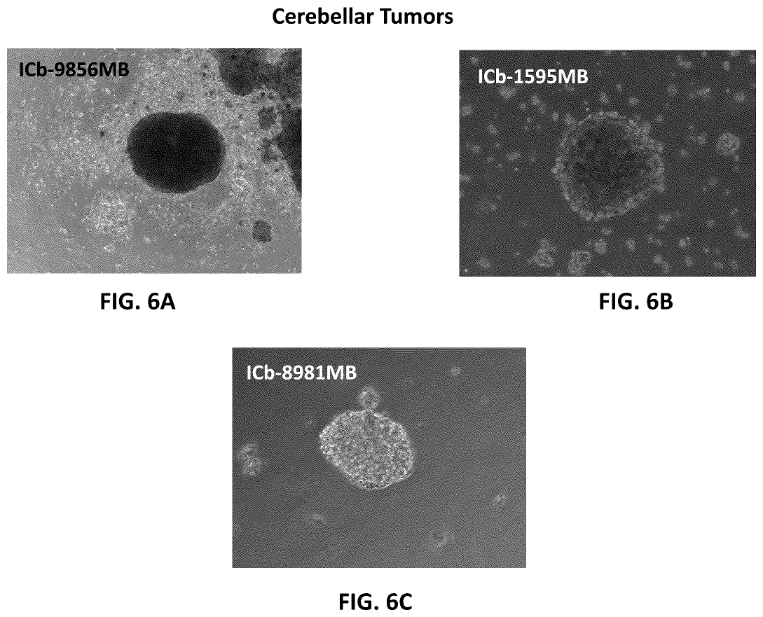

Brightfield images of organoids derived from single samples of cerebellar PDOX tumors (FIGS. 6A-6C). Cerebellar PDOX organoids' identifiers are listed in Table 4. Images of individual organoids were taken every day using the Keyence microscope and Zen (Carl Zeiss) software.

TABLE 10
Cerebellar Tumor Model IDs for FIG. 6

ICb-1595MB
ICb-0614MB
ICb-0832MB
ICb-8981MB
ICb-9856MB
ICb-3224MB
ICb-0409MB
ICb-5342LGG
ICb-5323EPN
ICb-2631AST
ICb-9810MB
ICb-5337MB
ICb-0174MB

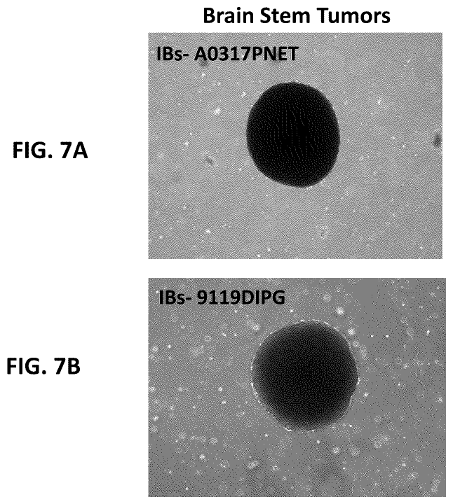

Brightfield images of organoids derived from single samples of brainstem PDOX tumors (FIGS. 7A-7B). Brainstem PDOX organoids' identifiers are listed in Table 5. Images of individual organoids were taken every day using the Keyence microscope and Zen (Carl Zeiss) software.

TABLE 11
Brain Stem Tumor Model IDs for FIG. 7

IBs-A0317PNET
IBs-2373PNET
IBs-9119DIPG
IBs-A052DIPG
IBs-A0405DIPG

### Example 5 Site-Related Media Facilitate Organoid Formation from PDOX Models

PDOX models are useful tools for developing and optimizing protocols for organoid model establishment. Differences on growth factor dependence for the formation of organoids from brain tumors of different locations results in increased success rate: 81.6% ( 40/49) success rate in PDOX models, and 90.9% ( 20/22) success rate in primary patient tumors. A mixed population of cells with the presence of cell proliferation on outer cellular layers and hypoxia/necrosis in the innermost cells of enlarging spheroids was successfully replicated. The disclosed targeted media formulations ensure high success rate of organoid formation, and biologically higher fidelity to human cancers, thereby significantly increasing the usefulness and/or predictability of preclinical drug testing for future clinical success. Further studies will analyze cellular and molecular characteristics of the organoids, with comparison with PDOX and/or patient tumor, and high-throughput drug testing using the developed organoids will be performed.

### Example 6 Cellular and Molecular Characterization of the Organoids

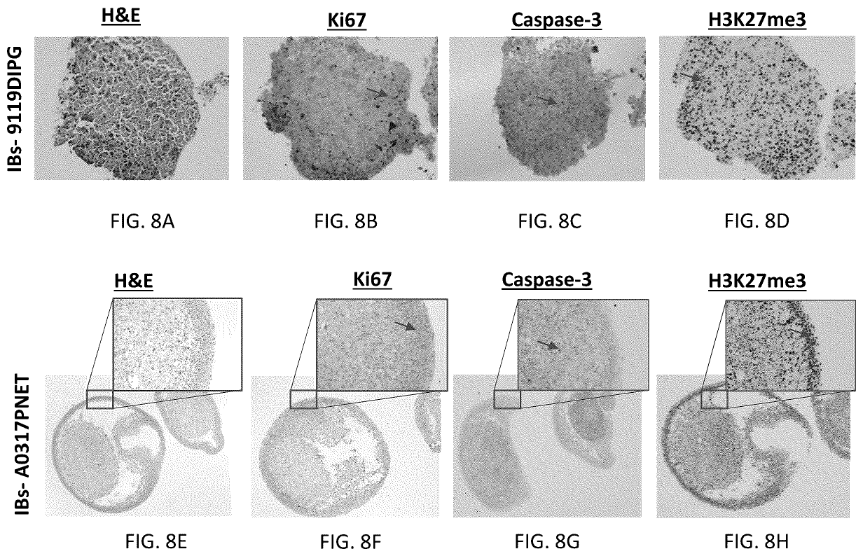

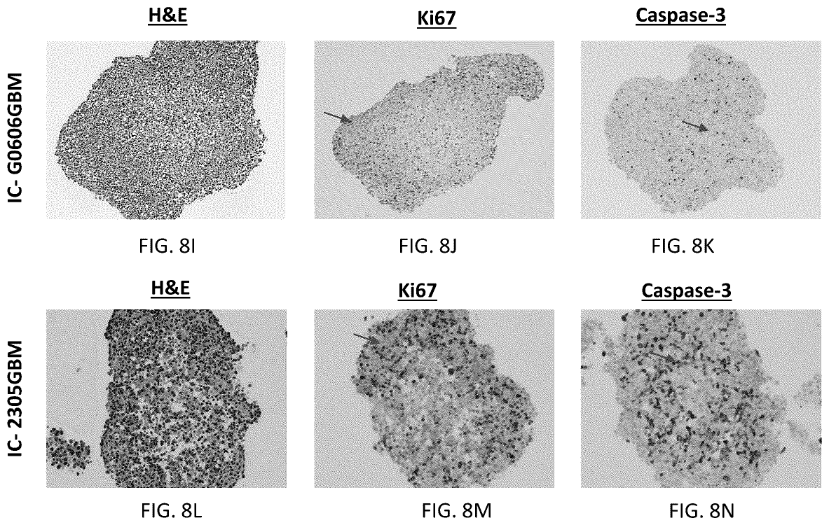

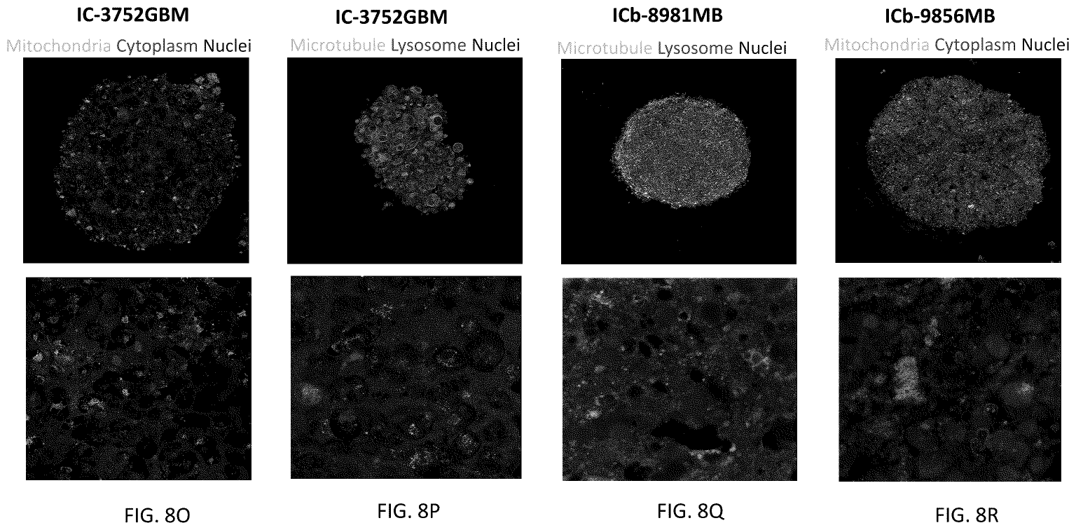

Cellular characterization studies of PDOX-derived organoids revealed replication of a heterogenous population. Immunohistochemistry for brainstem region (FIGS. 8A-8H) and cerebral (FIGS. 8I-8N) region using various organoid models revealed a mixed population of cells with the presence of cell proliferation on outer cellular layers and hypoxia/necrosis in the innermost cells of enlarging organoids closely resembling the nutrient and oxygen gradients found in tumors. Immunofluorescent characterization of cerebral organoid (FIGS. 8O-8P) and cerebellar organoid (FIGS. 8Q-8R) show differential expression of organelles (mitochondria, cytoplasm, lysosome, and nuclei) across the organoids consistent with the presence of an heterogenous cell population.

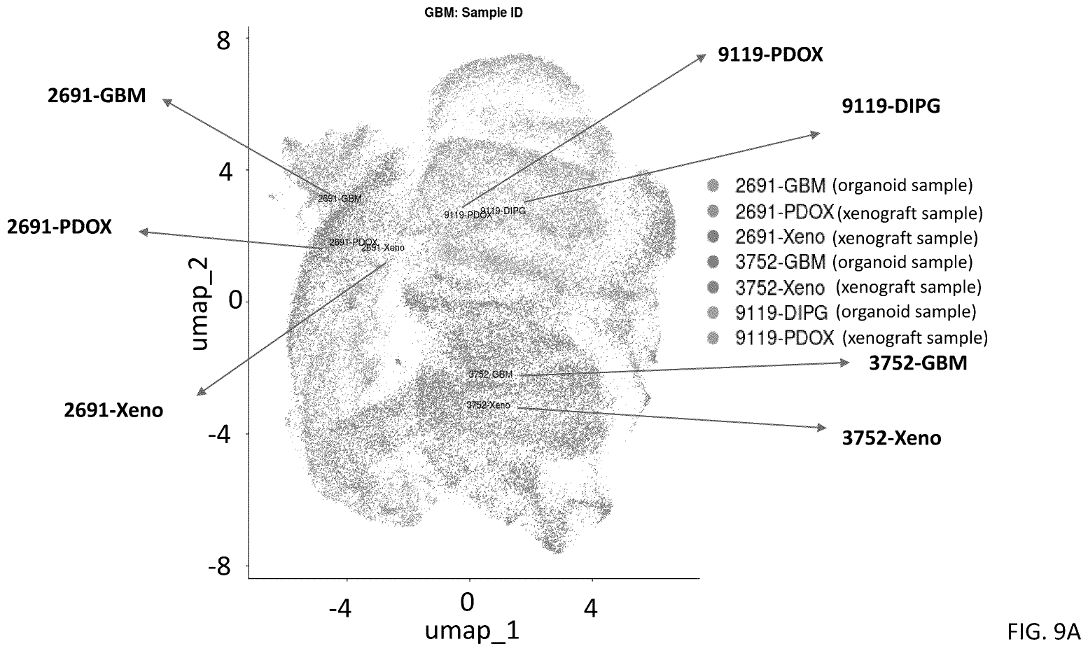

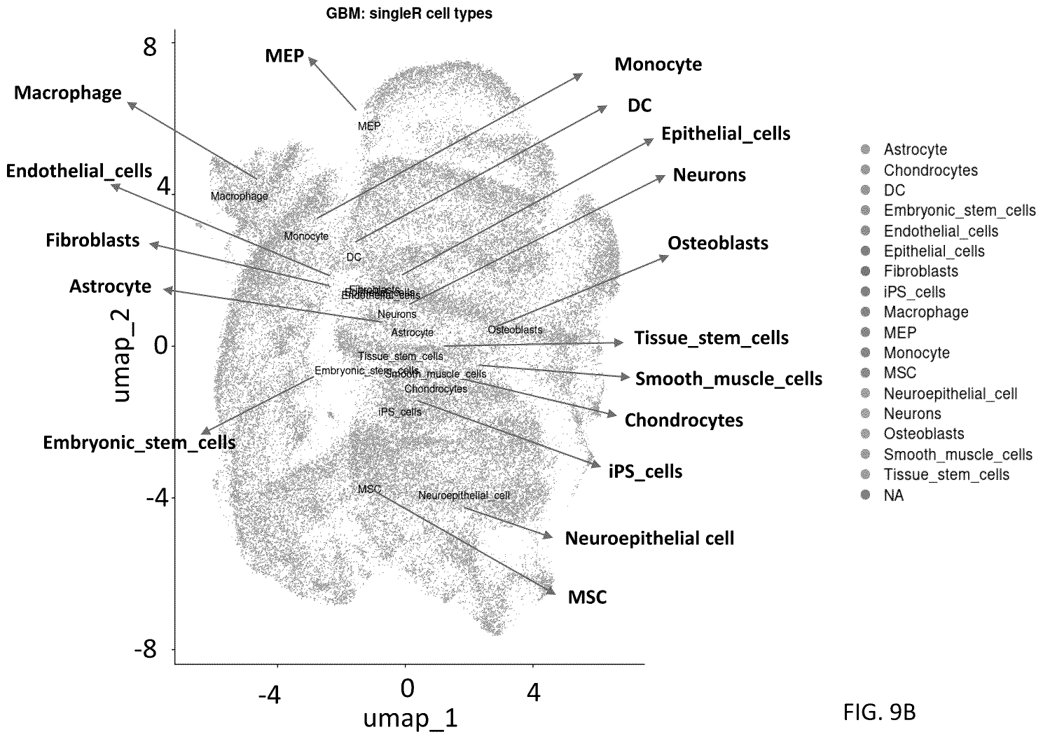

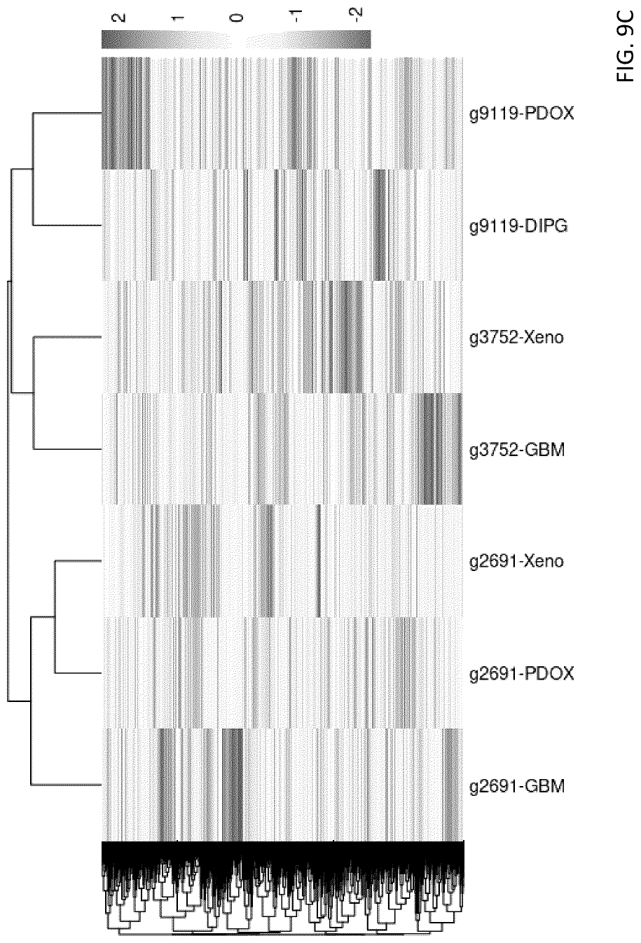

To investigate cell-type heterogeneity and its molecular signatures in the developed organoid models, the inventors performed single-cell RNA-sequencing of parental tumors from 3 patient-derived orthotopic xenografts (PDOX) samples and corresponding organoids (FIG. 9). Many different cell clusters were identified in parental tumors, reflecting the diversity of cell types and cellular states (FIGS. 9A-9B). Organoid clusters were mapped to the parental tumor cell clusters by pairwise comparisons of whole transcriptome gene expression with a high degree of similarity, indicating that the organoids largely maintain the cellular heterogeneity of parental tumors (FIG. 9C). Neoplastic populations of astrocyte-like, neuroepithelial-like, and neuron-like cells were identified (FIG. 9B). Together, single-cell RNA-sequencing analyses highlight marked cellular heterogeneity in organoids and further support that the developed organoids recapitulate cell-type heterogeneity and molecular signatures of corresponding parental tumors.

In describing the present invention and its various embodiments, specific terminology is employed for the sake of clarity. However, the invention is not intended to be limited to the specific terminology so selected. A person skilled in the relevant art will recognize that other equivalent components can be employed, and other methods developed without departing from the broad concepts of the current invention.

All patents and other publications cited anywhere in this specification are incorporated by reference in their entirety.

### REFERENCES

1. Lancaster, M. A. Brain organoids get vascularized. Nat. Biotechnol. 36, 407-408 (2018).
2. Lancaster, M. A. & Knoblich, J. A. Organogenesis in a dish: modeling development and disease using organoid technologies. Science 345, 1247125 (2014).
3. Lancaster, M. A. et al. Cerebral organoids model human brain development and microcephaly 1. Nature 501, 373-379 (2013).
4. Iefremova, V. et al. An Organoid-Based Model of Cortical Development Identifies Non-Cell Autonomous Defects in Wnt Signaling Contributing to Miller-Dieker Syndrome. Cell Rep. 19, 50-59 (2017).
5. Zhou, T. et al. High-Content Screening in hPSC-Neural Progenitors Identifies Drug Candidates that Inhibit Zika Virus Infection in Fetal-like Organoids and Adult Brain 4. Cell Stem Cell 21, 274-283 (2017).
6. Mariani, J. et al. Modeling human cortical development in vitro using induced pluripotent stem cells 1. Proc. Natl. Acad. Sci. U.S.A 109, 12770-12775 (2012).
7. Pasca, A. M. et al. Functional cortical neurons and astrocytes from human pluripotent stem cells in 3D culture 1. Nat. Methods 12, 671-678 (2015)

We claim:

1. A forebrain region tumor culture formulation comprising a basal medium supplemented with recombinant human basic fibroblast growth factor (FGF), recombinant human epidermal growth factor (EGF), brain-derived neurotrophic factor (BDNF), glial cell-derived neurotrophic factor (GDNF), neurotrophin 3 (NT-3), ciliary neurotrophic factor (CNTF), and wingless-relate integration site 3A (WNT-3A).

2. The forebrain region tumor culture formulation of claim 1, wherein the forebrain region tumor comprises cerebral cortex tumor.

3. A midbrain region tumor culture formulation comprising a basal medium supplemented with recombinant FGF-8 and recombinant sonic hedgehog (SHH).

4. The midbrain region cancer cell culture formulation of claim 3, wherein the midbrain region comprises tectum tumor or cerebral peduncle tumor.

5. A hindbrain region cancer cell culture formulation comprising a basal medium supplemented with recombinant FGF, recombinant FGF-19, and recombinant stromal cell-derived factor 1 alpha (SDF-1-α).

6. The hindbrain region cancer cell culture formulation of claim 5, wherein the hindbrain region comprises cerebellum tumor or pons tumor.

7. A hindbrain region cancer cell culture formulation comprising a basal medium supplemented with recombinant human basic recombinant FGF, recombinant human EGF, BDNF, GDNF, and NT-3.

8. The hindbrain region cancer cell culture formulation of claim 7, wherein the hindbrain region comprises brainstem tumor.

9. The formulation of claim 1, wherein the basal medium comprises serum-free DMEM/F12 Media supplemented with B27, N2, MEM-NEEAs, antibiotic, amino acid, 2-mercaptoethanol, and human insulin.

10. A method of forming brain region tumor organoids comprising producing a patient-derived orthotopic xenograft (PDOX) with forebrain region tumor and culturing the PDOX tumor in the formulation of claim 1 to form forebrain region patient-derived xenograph organoids (PDXOs).

11. A method of forming brain region tumor organoids comprising producing a patient-derived orthotopic xenograft (PDOX) with midbrain region tumor and culturing the PDOX tumor in the formulation of claim 3 to form midbrain region PDXOs.

12. A method of forming brain region tumor organoids comprising producing a patient-derived orthotopic xenograft (PDOX) with hindbrain region tumor and culturing the PDOX tumor in the formulation of claim 5 to form hindbrain region PDXOs.

13. A method of forming brain region tumor organoids comprising producing a patient-derived orthotopic xenograft (PDOX) with brainstem region tumor and culturing the PDOX tumor in the formulation of claim 7 to form brainstem region PDXOs.

14. The method of claim 10, wherein producing PDOX comprises obtaining brain tumor sample from the forebrain region, implanting the brain tumor sample into a matching location of a severe combined immunodeficient (SCID) mouse, and obtaining one or more forebrain region PDOX tumor sample from the mouse.

15. The method of claim 11, wherein producing PDOX comprises obtaining brain tumor sample from the midbrain region, implanting the brain tumor sample into a matching location of a severe combined immunodeficient (SCID) mouse, and obtaining one or more midbrain region PDOX tumor sample from the mouse.

16. The method of claim 12, wherein producing PDOX comprises obtaining brain tumor sample from the hindbrain region, implanting the brain tumor sample into a matching location of a severe combined immunodeficient (SCID) mouse, and obtaining one or more hindbrain region PDOX tumor sample from the mouse.

17. The method of claim 13, wherein producing PDOX comprises obtaining brain tumor sample from the brainstem region, implanting the brain tumor sample into a matching location of a severe combined immunodeficient (SCID) mouse, and obtaining one or more brainstem region PDOX tumor sample from the mouse.
